(19) United States
(12) Patent Application Publication
(10) Pub. No.: US 2013/0323283 A1
(43) Pub. Date: Dec. 5, 2013

(54) COMPOSITIONS AND METHODS FOR TREATING FOXP3+ TREG RELATED DISEASES

(71) Applicants: Wayne W. Hancock, Philadelphia, PA (US); Steven M. Albelda, Philadelphia, PA (US); Philip A. Cole, Baltimore, MD (US)

(72) Inventors: Wayne W. Hancock, Philadelphia, PA (US); Steven M. Albelda, Philadelphia, PA (US); Philip A. Cole, Baltimore, MD (US)

(73) Assignees: THE CHILDREN'S HOSPITAL OF PHILADELPHIA, Philadelphia, PA (US); THE JOHNS HOPKINS UNIVERSITY SCHOOL OF MEDICINE, Baltimore, MD (US); THE TRUSTEES OF THE UNIVERSITY OF PENNSYLVANIA, Philadelphia, PA (US)

(21) Appl. No.: 13/991,271

(22) PCT Filed: Dec. 1, 2011
(86) PCT No.: PCT/US11/62896

§ 371 Date: Aug. 9, 2013

Related U.S. Application Data
(60) Provisional application No. 61/418,552, filed on Dec. 1, 2010.

(51) Int. Cl. A61K 31/4155; A61K 38/16; A61K 31/7076; A61K 39/00

(52) U.S. Cl. CPC A61K 31/4155; A61K 39/0011; A61K 38/16; A61K 31/7076
USPC 424/277.1; 514/404; 424/94.3; 514/19.3; 514/47; 514/19.4; 514/19.5; 548/365.7; 530/326; 536/26.23; 435/32

(57) ABSTRACT

Methods for treating or preventing a Foxp3+ T regulatory cell (Treg) related disease in a subject in need thereof comprise administering to the subject an effective amount of a pharmaceutical composition comprising an inhibitor of a histone/protein acetyltransferase (HAT). Methods for identifying an agent useful for treating or preventing a Foxp3+ T regulatory cell (Treg) related disease comprise (a) contacting a candidate agent with a test sample comprising Foxp3+ T regulatory cells (Tregs), and (b) comparing a function of the Foxp3+ Tregs in the test sample with that in a control sample, wherein inhibition of the function of the Foxp3+ Tregs in the test sample when compared with the control sample indicates that the candidate agent is an agent useful for treating or preventing a Foxp3+ Treg related disease.

### CROSS-REFERENCE TO RELATED APPLICATION

This application claims the benefit of U.S. Provisional Application No. 61/418,552, filed Dec. 1, 2010, the contents of which are incorporated herein in their entireties for all purposes.

### FIELD OF THE INVENTION

The invention relates generally to compositions and methods for treating or preventing Foxp3+ T regulatory cell (Treg) related diseases. In particular, the invention relates to the use of histone/protein acetyltransferase (HAT) inhibitors to treat or prevent Foxp3+ Treg related diseases.

### BACKGROUND OF THE INVENTION

Cancers are a leading cause of death. For example, lung cancer continues to be the most common cause of cancer-related death. Screening for early detection has not reduced mortality. The 5-year survival rate (for all stages combined) is miserable—16%—even with the best that current surgery, radiation and chemotherapy can offer.

Historically, tumor growth and metastasis in the presence of an intact immune system were considered evidence of poor immunogenicity of tumor cells. In efforts to reduce the incidence of cancers, and especially recurrence after chemotherapy and/or radiation, investigators have repeatedly sought to increase the immunogenicity of cancers so as to promote host anti-cancer immune responses. These efforts have largely been unsuccessful.

The past decade has revealed the central importance of Foxp3+ T regulatory cells (Tregs) in regulating host immune responses and preventing autoimmunity, leading many to examine the possible role of Tregs in limiting anti-tumor responses. These data show that, especially in the case of solid tumors, the local accumulation of Foxp3+ Tregs is harmful and usually a negative prognostic indicator. Tantalizing experimental and limited clinical studies have shown that depleting Tregs or blocking their functions can boost anti-tumor T cell responses. However, current methods of Treg depletion have only transient efficacy and are often accompanied by increased risks of autoimmunity. Hence, the ability to decrease Treg function may be of major therapeutic significance if this can be done incrementally and without full-scale depletion of Tregs that are essential to maintenance of immune homeostasis and prevention of autoimmunity.

Histone/protein deacetylases (HDACs) catalyze removal of acetyl groups from lysines in histone tails and promote chromatin compaction and, typically, inhibit gene expression. In contrast, histone/protein acetyltransferases (HATs) promote acetylation and gene expression. HDACs and HATs also regulate acetylation of >1750 non-histone proteins. The effects of genetic or pharmacologic targeting of various classes of HATs on immune responses are largely unknown.

There remains a need for effective anti-cancer therapies by controlling host immune responses to cancers without concomitant suppression of conventional T cell responses.

### SUMMARY OF THE INVENTION

The present invention relates to methods for treating or preventing Foxp3+ T regulatory cell (Treg) related diseases by inhibiting Foxp3+ Treg functions, and related medicaments and compositions.

A method for treating or preventing a Foxp3+ T regulatory cell (Treg) related disease in a subject in need thereof is provided. The method comprises inhibiting a function of Foxp3+ Tregs in the subject.

A method for treating or preventing a Foxp3+ T regulatory cell (Treg) related disease in a subject in need thereof is also provided. The method comprises administering to the subject an effective amount of a pharmaceutical composition comprising an inhibitor of a histone/protein acetyltransferase (HAT). The pharmaceutical composition may further comprise a pharmaceutically acceptable carrier or diluent. The pharmaceutical composition may have a pH of 5.0-10.0.

A method for inhibiting the growth of a tumor in a subject in need thereof is provided. The method comprises inhibiting a function of Foxp3+ Tregs in the subject.

A method for inhibiting the growth of a tumor in a subject in need thereof is also provided. The method comprises administering to the subject an effective amount of a pharmaceutical composition comprising an inhibitor of a histone/protein acetyltransferase (HAT). The pharmaceutical composition may further comprise a pharmaceutically acceptable carrier or diluent. The pharmaceutical composition may have a pH of 5.0-10.0.

The method for treating or preventing a Foxp3+ T regulatory cell (Treg) related disease or inhibiting the growth of a tumor in a subject in need thereof may further comprise administering to the subject a cancer vaccine.

The Foxp3+ Treg related disease may be a cancer or tumor. The cancer may be selected from the group consisting of lung, ovary, endometrium, cervix, breast, prostate, head, neck, esophagus, stomach, liver, pancreas, colon, and skin (melanoma) cancers. The cancer may be a lung cancer.

The tumor may be a solid tumor. The solid tumor may be selected from the group consisting of lung, ovary, endometrium, cervix, breast, prostate, head, neck, esophagus, stomach, liver, pancreas, colon, and skin (melanoma) tumors.

The Foxp3+ Tregs may not be depleted in the subject. A function of effector T cells may not inhibited in the subject. The function of the effector T cells may be selected from the group consisting of T cell activation, T cell proliferation, and cytokine production.

The HAT inhibitor may inhibit a function of Foxp3+ Tregs. The Foxp3+ Tregs may be obtained from the subject. The HAT inhibitor may not inhibit a function of effector T cells. The function of the effector T cells may be T cell activation, T cell proliferation, or cytokine production. The effector T cells may be obtained from the subject.

The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300.

The HAT inhibitor may be selected from the group consisting of Lys-CoA, H3-CoA-20, C646 and functional derivatives. The HAT inhibitor may be C646.

A method for identifying an agent useful for treating or preventing a Foxp3+ T regulatory cell (Treg) related disease is provided. The method comprises (a) contacting a candidate agent with a test sample comprising Foxp3+ T regulatory cells (Tregs), and (b) comparing a function of the Foxp3+ Tregs in the test sample with that in a control sample. Inhibition of the function of the Foxp3+ Tregs in the test sample when compared with the control sample indicates that the candidate agent is an agent useful for treating or preventing a Foxp3+ Treg related disease. The Foxp3+ Treg related disease may be a cancer. The cancer may be selected from the group consisting of lung, ovary, endometrium, cervix, breast, prostate, head, neck, esophagus, stomach, liver, pancreas, colon and skin (melanoma) cancers. The cancer may be a lung cancer. The Foxp3+ Treg related disease may be a tumor. The Foxp3+ Treg related disease may be a tumor. The tumor may be a solid tumor selected from the group consisting of lung, ovary, endometrium, cervix, breast, prostate, head, neck, esophagus, stomach, liver, pancreas, colon and skin (melanoma) tumors.

A method for identifying an agent useful for inhibiting the growth of a tumor is also provided. The method comprises (a) contacting a candidate agent with a test sample comprising Foxp3+ T regulatory cells (Tregs), and (b) comparing a function of the Foxp3+ Tregs in the test sample with that in a control sample. Inhibition of the function of the Foxp3+ Tregs in the test sample when compared with the control sample indicates that the candidate agent is an agent useful for inhibiting the growth of the tumor. The tumor may be a solid tumor selected from the group consisting of lung, ovary, endometrium, cervix, breast, prostate, head, neck, esophagus, stomach, liver, pancreas, colon and skin (melanoma) tumors.

The test sample may further comprise effector T cells, and a function of the effector T cells may not be inhibited in the test sample when compared with that in the control sample. The function of the effector T cells may be selected from the group consisting of T cell activation, T cell proliferation, and cytokine production.

The test sample may be obtained from a subject who has suffered from the Foxp3+ T regulatory cell (Treg) related disease. The test sample may be obtained from a subject who is predisposed to the Foxp3+ T regulatory cell (Treg) related disease.

The agent useful for treating or preventing the Foxp3+ T regulatory cell (Treg) related disease or for inhibiting the growth of the tumor may be an inhibitor of a histone/protein acetyltransferase (HAT). The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300.

A medicament useful for treating or preventing a Foxp3+ T regulatory cell (Treg) related disease in a subject is provided. The medicament comprises an effective amount of an inhibitor of a histone/protein acetyltransferase (HAT). The Foxp3+ Treg related disease may be a cancer. The cancer may be selected from the group consisting of lung, ovary, endometrium, cervix, breast, prostate, head, neck, esophagus, stomach, liver, pancreas, colon and skin (melanoma) cancers. The cancer may be a lung cancer. The Foxp3+ Treg related disease may be a tumor. The tumor may be a solid tumor selected from the group consisting of lung, ovary, endometrium, cervix, breast, prostate, head, neck, esophagus, stomach, liver, pancreas, colon and skin (melanoma) tumors. The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300.

A medicament useful for inhibiting the growth of a tumor in a subject is also provided. The medicament comprises an effective amount of an inhibitor of a histone/protein acetyltransferase (HAT). The tumor may be a solid tumor selected from the group consisting of lung, ovary, endometrium, cervix, breast, prostate, head, neck, esophagus, stomach, liver, pancreas, colon and skin (melanoma) tumors. The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300.

The HAT inhibitor in the medicament of the present invention may inhibit a function of Foxp3+ Tregs. The Foxp3+ Tregs may be obtained from the subject. The HAT inhibitor may not inhibit a function of effector T cells. The function of the effector T cells may be selected from the group consisting of T cell activation, T cell proliferation, and cytokine production. The effector T cells may be obtained from the subject. The HAT inhibitor may be selected from the group consisting of Lys-CoA, H3-CoA-20, C646 and functional derivatives. The HAT inhibitor may be C646. The HAT inhibitor may have been identified by the identifying method of the present invention.

The medicament may further comprise a pharmaceutically acceptable carrier or diluent. The medicament may have a pH of 5.0-10.0.

A pharmaceutical composition for treating or preventing a Foxp3+ T regulatory cell (Treg) related disease in a subject is provided. The pharmaceutical composition comprises an effective amount of an inhibitor of a histone/protein acetyltransferase (HAT). The Foxp3+ Treg related disease may be a cancer. The cancer may be selected from the group consisting of lung, ovary, endometrium, cervix, breast, prostate, head, neck, esophagus, stomach, liver, pancreas, colon and skin (melanoma) cancers. The cancer may be a lung cancer. The Foxp3+ Treg related disease may be a tumor. The tumor may be a solid tumor selected from the group consisting of lung, ovary, endometrium, cervix, breast, prostate, head, neck, esophagus, stomach, liver, pancreas, colon and skin (melanoma) tumors. The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300.

A pharmaceutical composition for inhibiting the growth of a tumor in a subject is also provided. The pharmaceutical composition comprises an effective amount of an inhibitor of a histone/protein acetyltransferase (HAT). The tumor may be a solid tumor selected from the group consisting of lung, ovary, endometrium, cervix, breast, prostate, head, neck, esophagus, stomach, liver, pancreas, colon and skin (melanoma) tumors. The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300.

The HAT inhibitor in the pharmaceutical composition of the present invention may inhibit a function of Foxp3+ Tregs. The Foxp3+ Tregs may be obtained from the subject. The HAT inhibitor may not inhibit a function of effector T cells. The function of the effector T cells may be selected from the group consisting of T cell activation, T cell proliferation, and cytokine production. The effector T cells may be obtained from the subject. The HAT inhibitor may be selected from the group consisting of Lys-CoA, H3-CoA-20, C646 and functional derivatives. The HAT inhibitor may be C646. The HAT inhibitor may have been identified by the identifying method of the present invention.

The pharmaceutical composition of the present invention may further comprise a pharmaceutically acceptable carrier or diluent. The pharmaceutical composition may have a pH of 5.0-10.0.

A method of preparing a medicament useful for treating or preventing a Foxp3+ T regulatory cell (Treg) related disease in a subject is provided. The method comprises admixing an inhibitor of a histone/protein acetyltransferase (HAT) with a pharmaceutically acceptable carrier or diluent. The method may further comprise adjusting the pH of the medicament to 5.0-10.0. The Foxp3+ Treg related disease may be a cancer. The cancer may be selected from the group consisting of lung, ovary, endometrium, cervix, breast, prostate, head, neck, esophagus, stomach, liver, pancreas, colon and skin (melanoma) cancers. The cancer may be a lung cancer. The Foxp3+ Treg related disease may be a tumor. The tumor may be a solid tumor selected from the group consisting of lung, ovary, endometrium, cervix, breast, prostate, head, neck, esophagus, stomach, liver, pancreas, colon and skin (melanoma) tumors. The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300. The HAT inhibitor may inhibit a function of Foxp3+ Tregs. The Foxp3+ Tregs may be obtained from the subject. The HAT inhibitor may not inhibit a function of effector T cells. The function of the effector T cells may be T cell activation, T cell proliferation, or cytokine production. The effector T cells may be obtained from the subject. The HAT inhibitor may be selected from the group consisting of Lys-CoA, H3-CoA-20, C646 and functional derivatives. The HAT inhibitor may be C646. The HAT inhibitor may have been identified by the identifying method of the present invention.

A method of preparing a medicament useful for inhibiting the growth of a tumor in a subject is also provided. The method comprises admixing an inhibitor of a histone/protein acetyltransferase (HAT) with a pharmaceutically acceptable carrier or diluent. The method may further comprise adjusting the pH of the medicament to 5.0-10.0. The tumor may be a solid tumor selected from the group consisting of lung, ovary, endometrium, cervix, breast, prostate, head, neck, esophagus, stomach, liver, pancreas, colon and skin (melanoma) tumors. The HAT may be obtained from the subject. The HAT may be selected from the group consisting of GCN5, p300/CBP-associated factor (PCAF), Myst1, Myst2, Myst3, Myst4, TIP60, p300, and CBP. The HAT may be selected from the group consisting of GCN5, p300/CBP-associated factor (PCAF), Myst1, TIP60, p300, and CBP. The HAT may be p300. The HAT inhibitor may inhibit a function of Foxp3+ Tregs. The Foxp3+ Tregs may be obtained from the subject. The HAT inhibitor may not inhibit a function of effector T cells. The function of the effector T cells may be T cell activation, T cell proliferation, or cytokine production. The effector T cells may be obtained from the subject. The HAT inhibitor may be selected from the group consisting of Lys-CoA, H3-CoA-20, C646 and functional derivatives. The HAT inhibitor may be C646. The HAT inhibitor may have been identified by the identifying method of the present invention.

### BRIEF DESCRIPTION OF THE DRAWINGS

FIG. 1 shows that p300 binds to Foxp3 and promotes Foxp3 acetylation. Lysates of 293T cells cotransfected with Foxp3 and HA-tagged p300 expression vectors were immunoprecipitated with an anti-Foxp3 antibody. The Foxp3 immunoprecipitates (IP:Foxp3) were analyzed by Western blotting (WT) using an anti-acetylated lysine antibody (WB:Ac-K) or an anti-Foxp3 antibody (WB:Foxp3). Foxp3 is indicated with a star, and acetylated Foxp3 is indicated with an arrow (upper panel). Increasing the amount of the p300 expression vector led to increasing acetylation of Foxp3.

FIG. 2 shows gene expression of Foxp3, CTLA-4, GITR and TGF-β in Treg and Teff cells in the presence of DMSO (control) or 5 μM p300i (C646).

FIG. 3 shows inhibitory effects by p300i (C646) on Foxp3+ Treg functions in mice. (A) Gene expression of CTLA4, GITR, IL-10 and TGF-β in Treg and Teff cells from DMSO- or C646-treated mice. (B) Proliferation of Teff cells from DMSO-treated mice when mixed with Tregs from C646-treated mice at a Treg:Teff ratio of 2:1, 1:1, 1:2, 1:4, 1:8 or 0:1. (C) Proliferation of Teff cells from C646-treated mice when mixed with Tregs from DMSO-treated mice at a Treg:Teff ratio of 2:1, 1:1, 1:2, 1:4, 1:8 or 0:1.

FIG. 4 shows graft survival rates in cardiac allograft recipients receiving (A) 2:1 Teff:Treg cells along with C646 (p300i), CM-47 (p300i), H3-20-CoA-Tat (PCAFi), Lys-20-CoA-Tat (p300i), or DMSO; (B) Teff cells alone or with C646, or isographs with DMSO or C646; and (C) Teff cells alone or with (i) DMSO, (ii) Treg cells and DMSO, (iii) Treg cells and C646, or (iv) Treg cells and Lys-CoA-Tat (p300i).

FIG. 5 shows suppression of tumor growth in mice by (A) p300 depletion or (B) p300i (C646).

FIG. 6 shows inhibitory effects by C646 on gene expression and tumor growth in mice. (A) Gene expression of CD4, Foxp3, CD8 and Granzyme B mRNA in tumors from mice treated with DMSO or C646. (B) Tumor growth by volume (mm³) or by weight (g) in mice treated with DMSO or C646.

FIG. 7 shows lack of inhibitory effects by p300i (C646) on tumor growth in immunodeficient RAG−/− mice.

### DETAILED DESCRIPTION OF THE INVENTION

The present invention is based on the discovery of a central role for a histone/protein acetyltransferase (HAT), p300, in control of T regulatory cell (Treg) suppression. In particular, targeting p300 inhibits Treg functions in vitro and in vivo without concomitant suppression of T cell responses. The present invention relates generally to a new approach to cancer therapy allowing titratable and selective effects on host Foxp3+ Tregs vs. effector T cells by targeting p300 and other HATs. The present invention also relates to new tools for control of host immune responses to lung cancer and other types of cancers, in which Foxp3+ Tregs are thought to limit host immune responses and allow tumor growth.

The terms “protein” and “polypeptide” are used herein interchangeably, and refer to a polymer of amino acid residues with no limitation with respect to the minimum length of the polymer. The definition includes a full-length protein, and fragments or derivatives thereof. The fragments or derivatives preferably exhibit the same function as the protein. For example, a fragment or derivative of an enzyme may catalyze the same enzymatic reaction as the enzyme.

The term “fragment” of a protein as used herein refers to a polypeptide having an amino acid sequence that is the same as a part, but not all, of the amino acid sequence of the protein. The fragment may be a naturally occurring or recombinant molecule. The fragment may be unpurified or purified.

The term “derivative” of a protein used herein refers to a polypeptide having an amino acid sequence that is the same as the amino acid sequence of the protein except having at least one amino acid modified. Examples of the modifications include glycosylation, phosphorylation, methylation, acetylation, ubiquitination, deletions, additions and substitutions. The derivative may have an amino acid sequence at least about 80%, 90%, 95%, or 99%, preferably at least about 90%, more preferably at least about 95%, most preferably at least about 99%, identical to the amino acid sequence of the protein. The derivative may be a naturally occurring or recombinant molecule. The derivative may be unpurified or purified.

The present invention provides various methods, including methods for treating or preventing a Foxp3+ T regulatory cell (Treg) related disease in a subject in need thereof, and methods for inhibiting the growth of a tumor in a subject in need thereof. These methods comprise inhibiting a function of Foxp3+ Tregs in the subject. The survival rate may be improved, for example, by at least about 10%, 20%, 30%, 40%, 50%, 60%, 70%, 80%, 90%, or 100%, preferably by at least about 50%, more preferably by at least about 60%, over a time period of, for example, 1 day, 2 days, 3 days, 1 week, 2 weeks, 1 month, 2 months, 3 months, 6 months, 9 months, 1 year, 2 years or 5 years.

The term “Foxp3+ T regulatory cells (Tregs)” used herein refers to regulatory T cells expressing a Foxp3 protein. Foxp3 is expressed by CD4+CD25+ Tregs, and gain-of-function, overexpression and analysis of Foxp3-deficient Scurfy (sf) mice show Foxp3 is essential to the development and maintenance of murine Tregs. All naturally occurring murine CD4+CD25+ Treg cells express Foxp3. TGF-β1 can convert naïve CD4+CD25− T cells to CD4+CD25+ Tregs via induction of Foxp3. Unlike CTLA-4, GITR and CD25, murine Foxp3 mRNA expression appears stable irrespective of T cell activation. Various surface proteins (CTLA-4, GITR, LAG-3, neuropilin-1) and cytokines (TGF-β, IL-10) are expressed by Tregs, and like sf mice, mice lacking TGF-β, CTLA-4 or CD25 die from autoimmunity. Human X-linked neonatal diabetes mellitus, enteropathy and endocrinopathy (IPEX) syndrome results in most cases from mutations in the forkhead/winged-helix domain of FOXP3 that disrupt critical DNA interactions; in sf mice, a frameshift mutation results in a protein lacking the forkhead domain. More than 20 mutations of FOXP3 are reported in IPEX, and the syndrome is lethal if untreated. By contrast, overexpression of murine Foxp3 gene leads to hypocellular peripheral lymphoid tissues with fewer T cells and a hypoactive immune state. Hence, control of Foxp3 levels within a certain range is required for optimal immune functions and survival.

The term “a function of Foxp3+ Tregs” used herein refers to a suppressive function of Foxp3+ Tregs that relates to regulation of host immune responses and/or prevention of autoimmunity. A Foxp3+ Treg function may be suppression of an anti-tumor response by, for example, CD8+CD4+ T cells, natural killer (NK) cells, MØ, B cells, or dendritic cells (DCs), or suppression of proliferation of effector T cells.

While tumor cells have long been recognized to have distinct properties relating to growth, invasion and metastasis, their ability to resist and evade immune destruction is viewed as more complex than historical assessments that tumor cells lack sufficient antigenicity to promote a CD8+ T cell response. In particular, while the presence of tumor-infiltrating lymphocytes (TILs), especially CD8+ T cells, is typically associated with improved clinical outcome, the accumulation of Foxp3+ Tregs at the tumor site and/or in draining lymph nodes has a negative prognostic effect for many solid tumors.

The term “a Foxp3+ T regulatory cell (Treg) related disease” used herein refers to a disease or disorder linked to Foxp3+ T regulatory cells (Tregs). A Foxp3+ Treg related disease may be caused by a Fox3+Treg function, for example, suppression of an anti-tumor response or effector T cell proliferation. The Foxp3+ Treg related disease may be a cancer or a tumor.

A cancer may be any cancer. The cancer is preferably a lung (NSCLC), ovary, endometrium, cervix, breast, prostate, head, neck, esophagus, stomach, liver, pancreas, colon, or skin (melanoma) cancer, more preferably a lung cancer.

A tumor may be any tumor, preferably a solid tumor selected from the group consisting of lung (NSCLC), ovary, endometrium, cervix, breast, prostate, head, neck, esophagus, stomach, liver, pancreas, colon, and skin (melanoma) tumors.

A subject may be a mammal, for example, human, mouse, rat, horse, cattle (bovine), pig, sheep, goat, dog, and other domestic animals. Preferably, the subject is a human. The subject may have suffered from or may be predisposed to a Foxp3+ Treg related disease. Preferably, the subject has suffered from a Foxp3+ Treg related disease.

The subject may be a mouse, preferably a knockout mice, for example, a conditional knockout mice, as described in Kasper et al., Molecular and Cellular Biology (2006) 26(3): 789-809, the contents of which are incorporated in their entireties. The mouse may have a tumor. The tumor may be a solid tumor. Examples of solid tumors include, but are not limited to, lung, ovary, endometrium, cervix, breast, prostate, head, neck, esophagus, stomach, liver, pancreas, colon, and skin (melanoma) tumors.

The term “inhibiting a function of Foxp3+ Tregs” used herein refers to decreasing the level of the function, which may be determined by conventional techniques known in the art. The level of the function may be decreased by, for example, at least about 1%, 5%, 10%, 20%, 30%, 40%, 50%, 60%, 70%, 80%, 90%, 95%, 99% or 100%, preferably by at least about 50%, more preferably by at least about 90%, most preferably by at least about 100%. The inhibition of a Foxp3+ Tregs may be accomplished by various methods known in the art.

Experimentally, depletion of Foxp3+ Tregs is often beneficial in tumor bearing hosts, though various caveats apply. Strategies employed have ranged from the use of CD4 mAb or cyclophosphamide, to more direct targeting using CD25 or anti-CTLA4 mAb, or diphtheria or pseudomonas toxin conjugated to IL-2; and small molecule inhibitors or mAbs to disrupt signals promoting Treg development (e.g., COX-2/PGE2, TGF-β, aromatase inhibitors, STAT3i, TLR agonists, p38 MAPKi), recruitment (e.g., CCL17 or CCL22/CCR4) or function (e.g. blocking CTLA4, PD-1, GITR, IL-10, TGF-β). Additional strategies such as blockade of miRNA-155 or other miRNAs that regulate Treg development may also be envisioned.

However, common problems associated with the agents tested to date have been modest efficacy that may reflect co-targeting of activated effector T cells, increased rates of autoimmunity, inflammatory toxicity and only transient efficacy followed by rebound increases in Treg numbers. Additional concerns are the potential for promoting inflammation and thereby development of certain tumors, and possible depletion of Tregs that can have a favorable prognostic effects, such as in Hodgkin's lymphoma.

In the methods of the present invention, the Foxp3+ Tregs are preferably not depleted in the subject. The amount of Foxp3+ Tregs in the subject may be decreased by, for example, no more than about 10%, 20%, 30%, 40%, 50%, 60%, 70%, 80%, 90%, 95% or 99%, preferably by no more than about 50%, more preferably by no more than about 20%, most preferably by no more than about 10%.

In the methods of the present invention, a function of effector T cell function is preferably not inhibited in the subject. The level of the effector T cell function may be determined using conventional techniques known in the art, and may be decreased by, for example, no more than about 1%, 5%, 10%, 20%, 30%, 40%, or 50%, preferably by no more than about 10%, more preferably by no more than about 5%, most preferably by no more than about 1%. The effector T cell function may be T cell activation, T cell proliferation, or cytokine production.

The term “inhibiting the growth of a tumor” used herein refers to decreasing tumor growth, which may be determined by conventional techniques known in the art (e.g., by tumor weight or volume). The tumor growth may be caused by a Foxp3+ Treg function, for example, suppression of an anti-tumor response or effector T cell proliferation. The tumor growth may be decreased by, for example, at least about 1%, 5%, 10%, 20%, 30%, 40%, 50%, 60%, 70%, 80%, 90%, 95%, 99% or 100%, preferably by at least about 20%, more preferably by at least by about 50%, most preferably by at least about 70%, over a period of time up to about 1 day, 3 days, 5 days, one week, two weeks, one month, two months, three months, six months, nine months, one year or two years.

The methods according to the present invention may comprise administering to the subject an effective amount of a pharmaceutical composition comprising an inhibitor of a histone/protein acetyltransferase (HAT).

The term “a histone/protein acetyltransferase (HAT)” used herein refers to a full length protein capable of catalyzing acetylation of a histone or non-histone protein (e.g., Foxp3), or a functional fragment or derivative thereof. Acetylation of Foxp3 controls Treg development and suppressive activity, promoting chromatin binding and gene regulation in murine and human Tregs. The HAT may be a natural protein or a recombinant protein. A natural HAT may be obtained from a biological sample (e.g., a blood sample comprising T cells). The HAT may be obtained from a subject. The subject may have suffered from or be predisposed to a Foxp3+ Treg related disease. HATs are comprised of three super-families: GNAT (e.g., GCN5, and PCAF), MYST (e.g., Myst1/MOF, Myst2/HBO1, Myst3/MOZ, Myst4/MORF, and Tip60), and p300/CBP. Full length protein and gene sequences of various HATs in different species are known in the art. A recombinant HAT may be obtained using conventional techniques. A functional fragment or derivative of an HAT may retain the HAT acetylation activity, i.e., be capable of catalyzing acetylation of a histone or non-histone protein, preferably Foxp3.

The HAT may be selected from the group consisting of GCN5, p300/CBP-associated factor (PCAF), Myst1, Myst2, Myst3, Myst4, TIP60, p300, and CBP. Preferably, the HAT is GCN5, p300/CBP-associated factor (PCAF), Myst1, TIP60, p300, or CBP. More preferably, the HAT is p300.

The HAT activity may be measured by several different methods known in the art. For example, the HAT activity may be determined based on its ability to acetylate a substrate protein in vitro. The substrate protein may be a histone or non-histone protein, which may be known to be acetylated by the HAT. Preferably, the substrate protein is Foxp3. The substrate protein may be obtained from a subject, who may have suffered from or be predisposed to a Foxp3+ Treg related disease.

An HAT inhibitor may be an agent that is capable of decreasing the activity of an HAT. The agent may be a chemical compound or biological molecule. The biological molecule may be a nucleic acid molecule (e.g., siRNA and miRNA), a protein (e.g., antibody), or polypeptide (e.g., peptidic analogue). The HAT inhibitor may be associated with the HAT, and may have a Ki value of, for example, no more than about 1 mM, 500 μM, 100 μM, 10 μM, 1 μM, 750 nM, 500 nM, 400 nM, 200 nM, 100 nM or 10 nM, preferably no more than about 1 μM, more preferably no more than about 750 nM, most preferably no more than about 400 nM. The HAT activity may be decreased by the HAT inhibitor by, for example, at least about 10%, 20%, 30%, 40%, 50%, 60%, 70%, 80%, 90% or 95%, preferably by at least about 20%, more preferably by at least about 30%, most preferably by at least about 50%. The inhibition may be determined in vitro or in vivo using conventional techniques known in the art.

HAT inhibitors may be natural products such as curcumin, garcinol, anacardic acid, and plumbagin, or specific HAT inhibitors. The HAT inhibitors are preferably HAT specific inhibitors. Examples of HAT specific inhibitors include Lys-CoA, H3-CoA-20, C646 and functional derivatives thereof. Lys-CoA and H3-CoA-20 are peptidic inhibitors of p300/CBP and PCAF/GCN₅, respectively. C646 is a potent and selective small molecule active site inhibitor of p300/CBP with a Kᵢ of 400 nM, and is described in detail in Bowers et al., Chemistry & Biology (2010) 17, 1-12, the contents of which are incorporated in their entireties. More preferably, the HAT inhibitor is C646.

In accordance with the present invention, the HAT inhibitor may selectively inhibit a Foxp3+ Treg function. The HAT inhibitor preferably does not inhibit a function of effector T cells. The Foxp3+ Tregs and/or the effector cells may be obtained from the subject.

A desirable inhibition of a Foxp3+ Treg function may be achieved by adjusting the HAT inhibition incrementally by, for example, increasing the amount of a HAT specific inhibitor administered to the subject. The present invention allows titratable and selective effects on Foxp3+ Tregs vs. effector T cells in the subject.

A pharmaceutical composition for treating or preventing a Foxp3+ T regulatory cell (Treg) related disease or inhibiting the growth of a tumor in a subject comprises an effective amount of an inhibitor of a histone/protein acetyltransferase (HAT). The Foxp3+ Treg related disease may be a cancer or a tumor. The cancer may be a lung (NSCLC), ovary, endometrium, cervix, breast, prostate, head, neck, esophagus, stomach, liver, pancreas, colon, or skin (melanoma) cancer. Preferably, the cancer is a lung cancer. The tumor may be a solid tumor selected from the group consisting of lung (NSCLC), ovary, endometrium, cervix, breast, prostate, head, neck, esophagus, stomach, liver, pancreas, colon, and skin (melanoma) tumors.

The term “an effective amount” used herein refers to an amount of a pharmaceutical composition comprising an HAT inhibitor required to achieve a stated goal (e.g., treating or preventing a Foxp3+ Treg related disease or inhibiting the growth of a tumor in a subject in need thereof). The effective amount of the pharmaceutical composition comprising an HAT inhibitor may vary depending upon the stated goals, the physical characteristics of the subject, the nature and severity of the thrombotic disease, the existence of related or unrelated medical conditions, the nature of the HAT inhibitor, the composition comprising the HAT inhibitor, the means of administering the composition to the subject, and the administration route. A specific dose of an HAT inhibitor for a given subject may generally be set by the judgment of a physician. The pharmaceutical composition may be administered to the subject in one or multiple doses. Each dose may comprise an HAT inhibitor at about 0.001-5000 mg/kg, preferably about 0.01-1000 mg/kg, more preferably about 0.1-500 mg/kg. One or multiple doses may be administered to the subject per day.

The pharmaceutical composition may comprise about 0.01-20,000 μg, preferably about 0.1-1000 μg, more preferably about 0.5-500 μg of the HAT inhibitor. The pharmaceutical composition may comprise about 0.01-20,000 μg/ml, preferably about 0.1-1000 μg/ml, more preferably about 0.5-500 μg/ml.

The pharmaceutical composition may further comprise a pharmaceutically acceptable carrier or diluent. Carriers and diluents suitable in the pharmaceutical composition are well known in the art.

The pharmaceutical composition may have a pH of about 5.0-10.0, preferably about 5.6-9.0, more preferably about 6.0-8.8, most preferably about 6.5-8.0. For example, the pH may be about 6.2, 6.5, 6.75, 7.0, or 7.5.

The pharmaceutical compositions of the present invention may be formulated for oral, sublingual, intranasal, intraocular, rectal, transdermal, mucosal, topical or parenteral administration. Parenteral administration may include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, or intrathecal (spinal fluids) injection or infusion. Any device suitable for parenteral injection or infusion of drug formulations may be used for such administration. For example, the pharmaceutical composition may be contained in a sterile pre-filled syringe.

The methods of the present invention may further comprise administering to the subject a cancer vaccine. The subject may be predisposed to a Foxp3+ Treg related disease. The cancer vaccine may be administered to the subject before, during, or after, preferably before, administering to the subject the pharmaceutical composition comprising an HAT inhibitor.

The present invention also provides a method for identifying an agent useful for treating or preventing a Foxp3+ Treg related disease or for inhibiting the growth of a tumor. The method comprises (a) contacting a candidate agent with a test sample comprising Foxp3+ Tregs, and (b) comparing a function of the Foxp3+ Tregs in the test sample with that in a control sample. A decrease in the Foxp3+ Treg function in the test sample when compared with that in the control sample indicates that the candidate agent is useful for treating or preventing a Foxp3+ Treg related disease or inhibiting the growth of a tumor. The level of the Foxp3+ Treg function in the test sample may be decreased by, for example, at least about 50%, 60%, 70%, 80%, 90%, 95%, 99% or 100%, preferably by at least about 50%, more preferably by at least by about 90%, most preferably by at least about 100%, when compared with that in the control sample.

The test sample may be a biological sample, comprising, for example, cells and/or tissues. Preferably, the test sample is a blood sample obtained from a subject. The subject may have suffered from or may be predisposed to the Foxp3+ Treg related disease. More preferably, the test sample is obtained from a subject who has suffered from the Foxp3+ Treg related disease.

In some embodiments, the test sample may further comprise effector cells, and an effector cell function may not be inhibited in the test sample when compared with that in the control sample. The level of the effector T cell function in the test sample may be decreased by, for example, no more than about 1%, 5%, 10%, 20%, 30%, 40%, or 50%, preferably by no more than about 10%, more preferably by no more than about 5%, most preferably by no more than about 1%. The effector cell function may be selected from the group consisting of T cell activation, T cell proliferation, and cytokine production.

The control sample is similar to the test sample, but has not been contacted with the candidate agent. The control sample may be the same as the test sample except that it has not been contacted with the candidate agent. For example, the control sample may be the test sample before being contacted with the candidate agent.

The agent identified as useful for treating or preventing a Foxp3+ Treg related disease or inhibiting the growth of a tumor may be an inhibitor of an HAT. The HAT may be a natural protein or recombinant protein. In some embodiments, the HAT is obtained from Foxp3+ Tregs. The Foxp3+ Tregs may be obtained from a subject. The subject may have suffered from a Foxp3+ Treg related disease. The Foxp3+ Treg related disease may be a cancer or tumor. The cancer may be a lung (NSCLC), ovary, endometrium, cervix, breast, prostate, head, neck, esophagus, stomach, liver, pancreas, colon, or skin (melanoma) cancer. Preferably, the cancer is a lung cancer. The tumor may be a solid tumor selected from the group consisting of lung (NSCLC), ovary, endometrium, cervix, breast, prostate, head, neck, esophagus, stomach, liver, pancreas, colon, and skin (melanoma) tumors. The HAT inhibitor identified according to this method may be used in the method for treating or preventing a Foxp3+ Treg related disease or inhibiting the growth of a tumor in a subject in need thereof.

The present invention further provides a medicament useful for treating or preventing a Foxp3+ Treg related disease or for inhibiting the growth of a tumor in a subject. It comprises an effective amount of an inhibitor of an HAT. The Foxp3+ Treg related disease may be a cancer or a tumor. The cancer may be a lung (NSCLC), ovary, endometrium, cervix, breast, prostate, head, neck, esophagus, stomach, liver, pancreas, colon, or skin (melanoma) cancer. Preferably, the cancer is a lung cancer. The tumor may be a solid tumor selected from the group consisting of lung (NSCLC), ovary, endometrium, cervix, breast, prostate, head, neck, esophagus, stomach, liver, pancreas, colon, and skin (melanoma) tumors.

With respect to the HAT inhibitor in the medicament of the present invention, the HAT may be a full length protein capable of catalyzing acetylation of a histone or non-histone protein (e.g., Foxp3), or a functional fragment or derivative thereof. The HAT may be a natural protein or a recombinant protein. A natural HAT may be obtained from a biological sample (e.g., a blood sample comprising T cells) or from a subject. The HAT may be selected from the group consisting of GCN5, p300/CBP-associated factor (PCAF), Myst1, Myst2, Myst3, Myst4, TIP60, p300, and CBP. Preferably, the HAT is GCN5, p300/CBP-associated factor (PCAF), Myst1, TIP60, p300, or CBP. More preferably, the HAT is p300.

The HAT inhibitor in the medicament of the present invention may be a natural or recombinant protein. It may inhibit a function of Foxp3+ Tregs, which may be obtained from the subject. The HAT inhibitor may decrease the level of the Foxp3+ Treg function by, for example, at least about 50%, 60%, 70%, 80%, 90%, 95%, 99% or 100%, preferably by at least about 50%, more preferably by at least about 90%, most preferably by at least about 100%.

The HAT inhibitor in the medicament of the present invention preferably does not inhibit a function of effector T cells. The HAT inhibitor may decrease the level of the effector T cell function by, for example, no more than about 1%, 5%, 10%, 20%, 30%, 40%, or 50%, preferably by no more than about 10%, more preferably by no more than about 5%, most preferably by no more than about 1%. The effector cell function may be selected from the group consisting of T cell activation, T cell proliferation, and cytokine production. The effector T cells may be obtained form the subject.

The HAT inhibitor in the medicament of the present invention may be any agent that is capable of decreasing the activity of an HAT. The agent may be a chemical compound or biological molecule. The biological molecule may be a nucleic acid molecule (e.g., siRNA or miRNA), a protein (e.g., antibody), or polypeptide (e.g., peptidic analogue). The HAT inhibitor may be associated with the HAT, and may have a Ki value of, for example, no more than about 1 mM, 500 μM, 100 μM, 10 μM, 1 μM, 750 nM, 500 nM, 400 nM, 200 nM, 100 nM or 10 nM, preferably no more than about 1 μM, more preferably no more than about 750 nM, most preferably no more than about 400 nM. The HAT activity may be decreased by the HAT inhibitor by, for example, at least about 10%, 20%, 30%, 40%, 50%, 60%, 70%, 80%, 90% or 95%, preferably by at least about 20%, more preferably by at least about 30%, most preferably by at least about 50%. The inhibition may be determined in vitro or in vivo using conventional techniques known in the art. Examples of HAT inhibitors include natural products (e.g., curcumin, garcinol, anacardic acid, and plumbagin) and specific HAT inhibitors (e.g., Lys-CoA, H3-CoA-20, C646 and functional derivatives thereof). Preferably, the HAT inhibitor is a specific HAT inhibitor. More preferably, the HAT inhibitor is C646. The HAT inhibitor may have been identified in accordance with the present invention.

The medicament may further comprise a pharmaceutically acceptable carrier or diluent. The pH of the medicament may be in the range of about 5.0-10.0, preferably about 5.6-9.0, more preferably about 6.0-8.8, most preferably about 6.5-8.0.

For each medicament of the present invention, a method for preparing the medicament is provided. The preparation method comprises admixing an inhibitor of an HAT with a pharmaceutically acceptable carrier or diluent. The method may further comprise adjusting the pH of the medicament to about 5.0-10.0, preferably about 5.6-9.0, more preferably about 6.0-8.8, most preferably about 6.5-8.0. The Foxp3+ Treg related disease may be a cancer or a tumor. The cancer may be a lung (NSCLC), ovary, endometrium, cervix, breast, prostate, head, neck, esophagus, stomach, liver, pancreas, colon, or skin (melanoma) cancer. Preferably, the cancer is a lung cancer. The tumor may be a solid tumor selected from the group consisting of lung (NSCLC), ovary, endometrium, cervix, breast, prostate, head, neck, esophagus, stomach, liver, pancreas, colon, and skin (melanoma) tumors. The HAT may be obtained from a subject. The HAT may be selected from the group consisting of GCN5, p300/CBP-associated factor (PCAF), Myst1, Myst2, Myst3, Myst4, TIP60, p300, and CBP. Preferably, the HAT is GCN5, p300/CBP-associated factor (PCAF), Myst1, TIP60, p300, or CBP. More preferably, the HAT is p300. The HAT inhibitor may be Lys-CoA, H3-CoA-20, C646 or a functional derivative thereof. Preferably, the HAT inhibitor is C646. The HAT inhibitor may have been identified in accordance with the present invention. The HAT inhibitor may inhibit a function of Foxp3+ Tregs, which may be obtained from the subject. Preferably, the HAT inhibitor does not inhibit a function of effector T cells, which may be obtained from the subject. The effector cell function may be T cell activation, T cell proliferation, or cytokine production.

### Example 1

### p300/CBP are Key HATs in Tregs

p300 is expressed by Tregs, and co-localized with Foxp3+ in the nuclei of murine Foxp3+ Treg. Comparable co-localization of p300 and Foxp3 was observed in transfected 293T cells.

Foxp3 gene expression by Tregs from wild-type (WT) mice, p300−/− (floxed p300) mice and CBP−/− (floxed CBP) mice was compared. The floxed p300 and floxed CBP mice were obtained as described in Kasper et al., Mol. Cell Biol. (2006) 26:789-809, the contents of which are incorporated in their entireties. Microarray data shows that p300 or CBP deletion led to down-regulation of Foxp3 gene expression.

### Example 2

### p300 Binds to Foxp3 and Promotes Foxp3 Acetylation

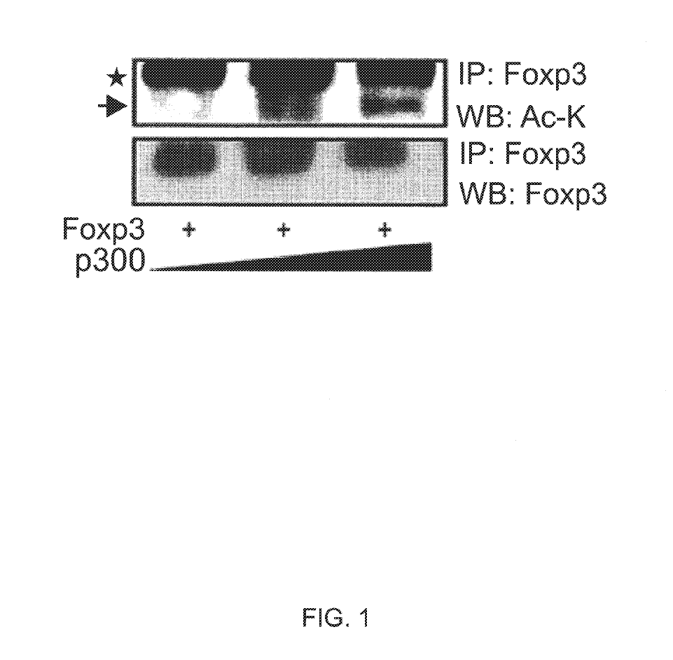

Biochemical studies using 293T cells co-transfected with Foxp3 and HA-tagged p300 were carried out to assess the interaction between Fox3 and p300. Immunoprecipitation of p300 resulted in co-precipitation of Foxp3, and immunoprecipitation of Foxp3 likewise led co-precipitated p300. This interaction was of functional importance, since cotransfection of 293T cells with Foxp3 and p300 led to acetylation of Foxp3, as observed by immunoprecipitation of Foxp3 (IP: Foxp3) and Western blotting for acetylated lysine (WB: Ac-K) (FIG. 1), and increasing levels of p300 led to increasing acetylation of Foxp3 (FIG. 1). C646 was found to impair Foxp3 acetylation. These studies show that p300 can physically interact with, and acetylate, Foxp3.

### Example 3

### Treg Suppression Impaired by C646 In Vitro

The effects of C646 on Treg suppression of effector T cell proliferation were evaluated in standard in vitro murine Treg suppression assays, performed as described in Tao et al., Nat. Med. (2007) 13:1299-307, the contents of which are incorporated by their entireties. In these assays, effector T cells (Teffs) were labeled with carboxyfluorescein diacetate succinimidyl ester (CFSE), and stimulated by CD3 mAb plus irradiated antigen presenting cells (APCs). Tregs and Teffs were mixed at a ratio of 0:1, 1:8, 1:4, 1:2 or 1:1, and cultured in the presence of DMSO (control) or 5 μM C646 (HATi). The percentage of proliferating CFSE-labeled Teffs in each mixture was assessed by flow cytometry after 72 hours as shown in Table 1.

TABLE 1
Effect of C646 on effector T cell proliferation

| Treg:Teff | 0:1 | 1:8 | 1:4 | 1:2 | 1:1 |
|---|---|---|---|---|---|
| DMSO (% proliferating cells) | 89% | 48% | 35% | 24% | 18% |
| C646 (% proliferating cells) | 88% | 85% | 78% | 70% | 66% |

In the absence of Tregs (i.e., Treg:Teff ratio of 0:1), the percentages of proliferation of murine Teff was comparable for DMSO (control) treatment or C646 (HATi) treatment, whereas addition of Tregs plus DMSO led to a stepwise impairment of proliferation as the ratio of Tregs to Teff cells increased. Comparison of the DMSO and C646 results shows that addition of the HATi (5 μM) largely abrogated the suppressive actions of Tregs in this system. Similar data was observed using (i) Tregs incubated with HATi and then washed pre-assay, and (ii) with Lys-CoA-Tat, which is an HAT inhibitor. These studies show that inhibition of p300/CBP can largely erase the suppressive functions of Tregs in vitro and yet leave CD3 mAb-induced T cell proliferation unimpaired.

### Example 4

### Treg Gene Expression Impaired by C646 In Vitro

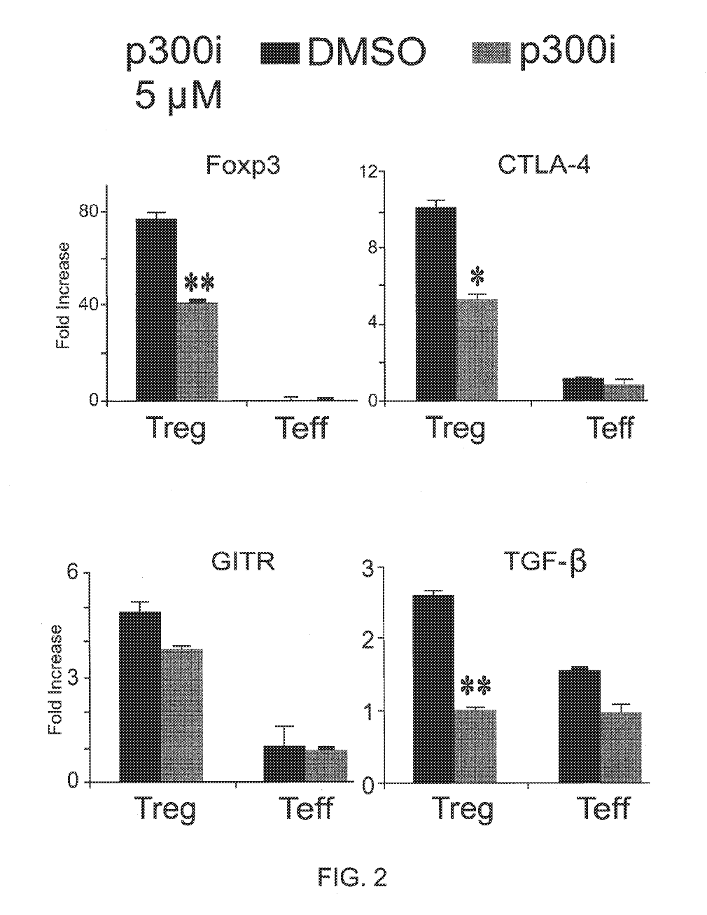

The effects of C646 on Treg gene expression of Foxp3, CTLA-4, GITR and TGF-β were assessed. Treg and Teff cells were activated for 24 hours with CD3/CD28 mAbs plus IL-2 in the presence of 5 μM C646 (p300i) or DMSO alone (control). Gene expression Foxp3, CTLA-4, GITR and TGF-β in the activated Treg and Teff cells was determined by qPCR (mean±SD, n=3/grp) and normalized to 18S. C646 downregulated Treg expression of Foxp3, CTLA-4, and TGF-β genes with a statistical significance, and GITR gene (FIG. 2).

### Example 5

### CD4+ Foxp3+ Proportion in Teffs and Tregs Impaired by C646 In Vitro

The effects of C646 (p300i) on the CD4+ Foxp3+ proportion in Teffs and Tregs were assessed. Teffs and Tregs were isolated, and treated with 5, 10, or 20 μM p300i or DMSO for 6 hours. Flow cytometry was used to determine the CD4+ Foxp3+ proportion in Teffs or Tregs (Table 2) and the mean fluorescent intensity (MFI) of CD4+ Foxp3+ cells in Teffs or Tregs (Table 3) before or after being treated with p300i or DMSO. These data demonstrate that p300i use decreases the CD4+Foxp3+ proportion and MFI in Tregs.

TABLE 2
Effect of C646 on CD4+ Foxp3+ proportion

| | | |
|---|---|---|
| Concentration | Teffs | Tregs |

| (μM) | DMSO | C646 | DMSO | p300i |
|---|---|---|---|---|
| 0 | 2 | 2 | 89 | 89 |
| 5 | 2 | 2 | 75 | 71 |
| 10 | 2 | 2 | 76 | 68 |
| 20 | 2 | 1 | 75 | 49 |

TABLE 3
Effect of C646 on mean fluorescent intensity
(MFI) of CD4+ Foxp3+ cells

| | | |
|---|---|---|
| Concentration | Teff | Treg |

| (μM) | DMSO | C646 | DMSO | C646 |
|---|---|---|---|---|
| 0 | 111 | 111 | 191 | 191 |
| 5 | 131 | 121 | 273 | 280 |
| 10 | 109 | 108 | 306 | 253 |
| 20 | 81 | 91 | 301 | 138 |

### Example 6

### Foxp3+ Treg Suppression and Gene Expression Impaired by C646 In Vivo

The effects of C646 (p300i) on T cells in vivo were studied. DMSO or C646 was administered to B6 mice at 0.9 mg/kg/day via Alzet pump for 7 days. Treg and Teff cells were purified using magnetic beads from DMSO- or p300i-treated mice.

The proportions of CD4, CD8, Foxp3+ CD4, or CD62L CD44 (markers of memory vs. naive T cells) cells in lymph nodes (LN) and spleen (SP) of DMSO- and C646-treated mice were determined as shown in Table 4. Compared to DMSO-treated mice, mice given C646 had negligible effect on the proportions of the tested cells.

TABLE 4
Effect of C646 on CD4, CD8, Foxp3+
CD4, and CD62L CD44 proportions

| | |
|---|---|
| LN | SP |

| Cells | DMSO | C646 | DMSO | C646 |
|---|---|---|---|---|
| CD4 | 36 | 35 | 17 | 18 |
| CD8 | 30 | 34 | 10 | 12 |
| Foxp3+ CD4 | 8 | 8 | 13 | 13 |
| CD62L CD44 | 5 | 6 | 15 | 12 |

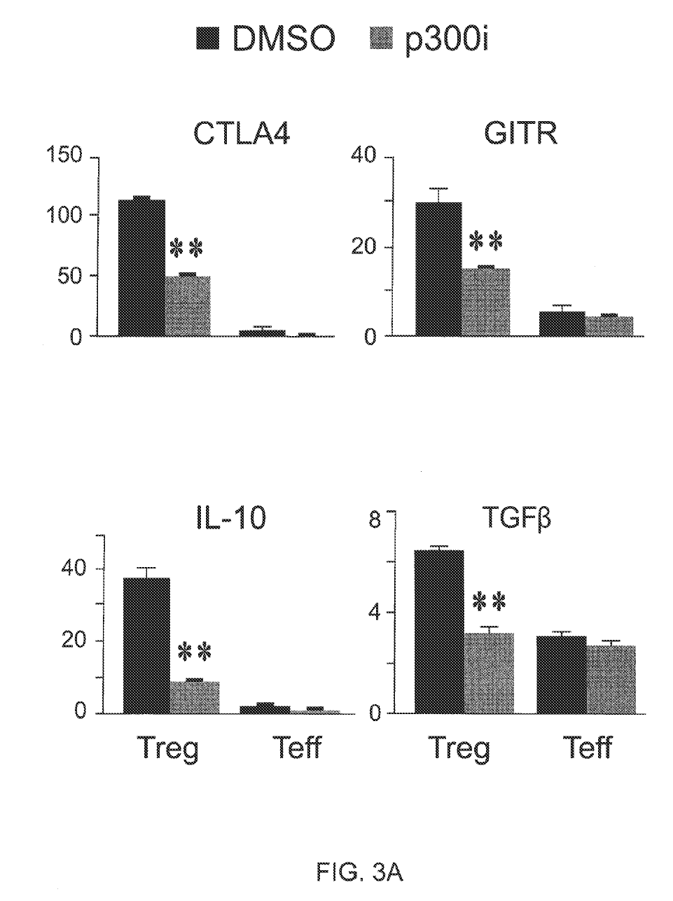

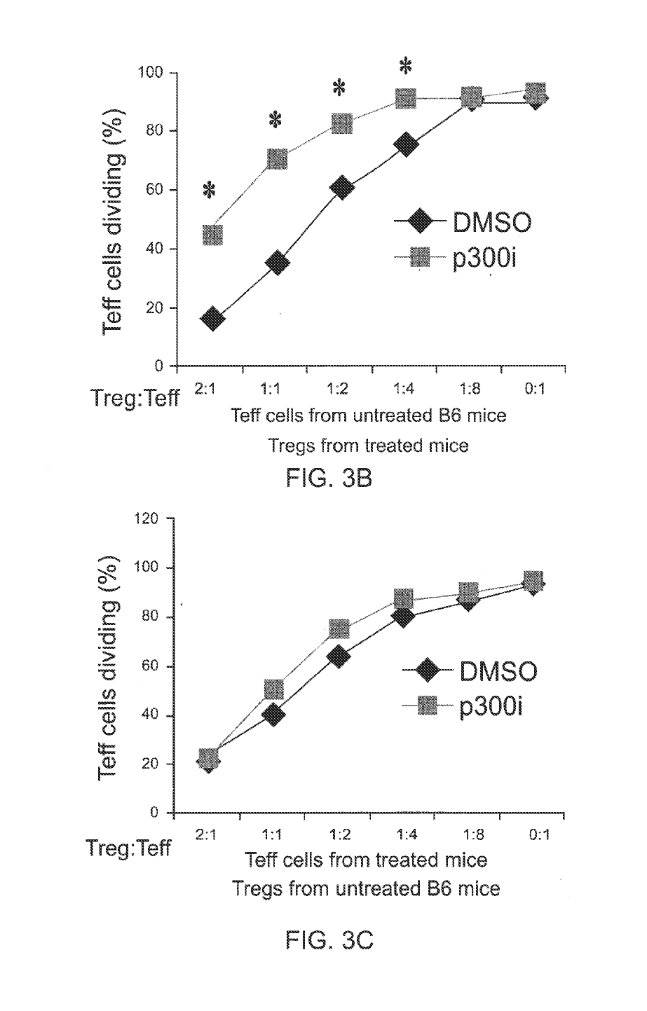

Gene expression by Treg and Teff cells from DSMO- or C646-treated mice (four mice/group) was analyzed by qPCR. C646 significantly decreased (**p<0.01) expression of multiple Foxp3-associated genes, CTLA4, GITR, IL-10 and TGF-β, by Treg cells, whereas expression of these genes by Teff cells was unchanged (p>0.05) (FIG. 3A).

Tregs and Teff cells were purified from DMSO-treated mice (untreated) or C646-treated mice, and used in Treg suppression assays to assess the effects of C646 on Treg suppression of proliferation of Teff cells. When Tregs from C646-treated mice were mixed with Teff cells from untreated mice at a ratio of 2:1, 1:1, 1:2, 1:4, 1:8 or 0:1, C646 use decreased the ability of Tregs to suppress proliferation of Teff cells from untreated mice (FIG. 3B, *p<0.05). When Tregs from untreated mice were mixed with Teff cells from treated mice at a ratio of 2:1, 1:1, 1:2, 1:4, 1:8 or 0:1, the Teff cells from C646 treated mice responded normally to suppression by control normal Tregs (FIG. 3C, *p<0.05).

The data are representative of three independent experiments (*p<0.05, **p<0.01). The data demonstrate that p300 targeting impairs Treg functions, including gene expression and suppression of Teff cell proliferation, without affecting those of Teffs.

### Example 7

### Effector T Cells not Impaired by C646 in Allograft Recipients

The effect of C646 on alloantigen-induced T cell proliferation in vivo was evaluated in two parent-to-F1 assays.

In the first parent-to-F1 assay (C57BL/6->DBA/B6) (Tao et al., J. Immunol. (2005) 175:5774-82; Tao et al., J. Immunol. (2008) 180:6649-55; the contents of both of which are incorporated herein in their entireties), activation and proliferation of adoptively transferred CFSE-labeled CD4 and CD8 cells was monitored by flow cytometry in a 3 day assay. DMSO (control) or C646 was administered at 0.8 mg/kg/d with continuous delivery via Alzet pump. The percentages of CFSE-labeled CD4 proliferating or dividing cells from DSMO- and C646-treated mice were 65% and 73%, respectively. The percentages of CFSE-labeled CD8 proliferating or dividing cells from DSMO- and C646-treated mice were 93% and 95%, respectively. C646 administration did not impair alloantigen-induced T cell activation or proliferation (CFSE dilution) or production of cytokines such as IL-2 and IFN-γ.

In the second parent-to-F1 assay, CFSE-labeled T cells from C57BL/6 (B6) mice were injected into B6D2F1 mice, and the recipients were treated with DMSO (control) or C646 (p300i) at 0.9 mg/kg/d for 3 days by Alzet pumps. The percentages of CFSE-labeled CD4 proliferating cells from DSMO- and C646-treated mice were 94.5% and 95.0%, respectively. The percentages of CFSE-labeled CD8 proliferating or dividing cells from DSMO- and C646-treated mice were 78.5% and 81.6%, respectively. Donor CD4 and CD8 cells (H-2d negative) from both groups had similar alloantigen-induced activation and proliferation (p>0.05).

These data demonstrate that p300i (C646) does not inhibit normal effector T cell responses or affect T cell alloactivation in vivo.

### Example 8

### Treg Function Blocked by HATi In Vivo

The effects of several HAT inhibitors (HATi) in a cardiac allograph model were studied in three experiments. This model involved vascularized cardiac allografts from BALB/c donors transplanted into immunodeficient C57BL/6 RAG−/− (B6 RAG−/−) recipients. The recipients were adoptively transferred with 1×10⁶ purified C57BL/6 Teff cells (CD4+CD25−) alone or in combination with 0.5×10⁶ purified Treg cells (CD4+CD25+). The cardiac allograft survival rates of the allograft recipients were monitored over a period of 120 days post-transplant. In this model, intravenous injection of 1×10⁶ purified C57BL/6 Teff cells (CD4+CD25−) induced cardiac allograft rejection in 10-12 days, whereas co-transfer of 1×10⁶ Teff cells and 0.5×10⁶ purified Tregs (CD4+CD25+) resulted in long-term (>100 d) cardiac allograft survival.

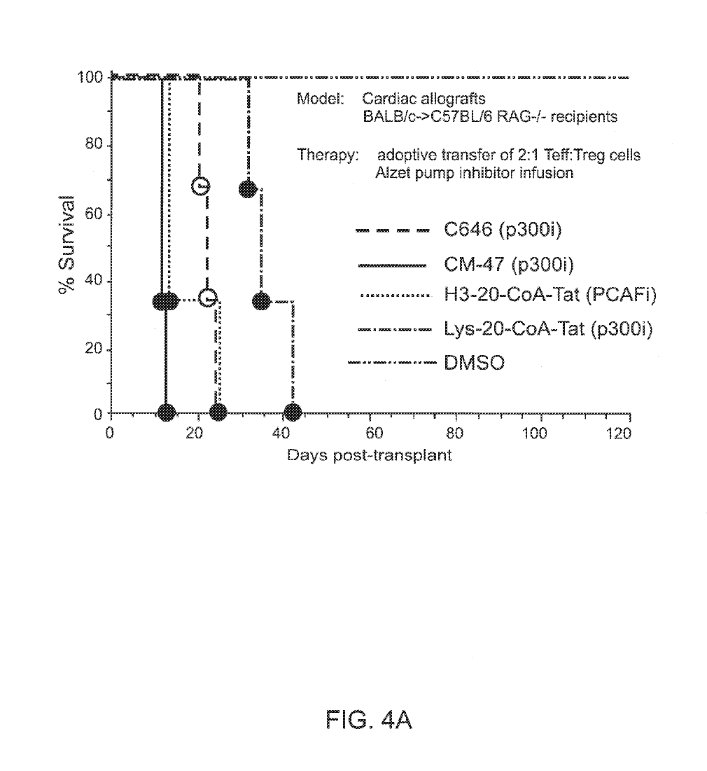

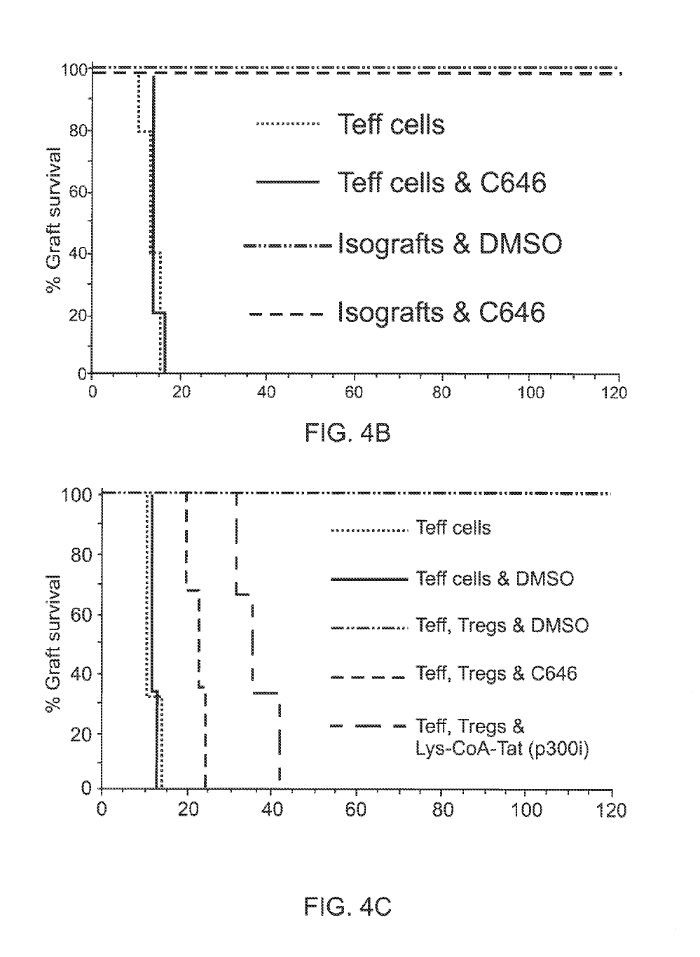

In the first experiment, the allograft recipients were adoptively transferred with 1×10⁶ Teff cells and 0.5×10⁶ purified Tregs (CD4+CD25+), and treated with DMSO (control) or a HAT inhibitor via Alzet pump. Four HAT inhibitors were tested. Three were p300/CBP inhibitors, C646, a related compound CM-47 and the peptidic HAT1, H3-CoA-20-Tat. One was a PCAF/GCN5 inhibitor, Lys-20-CoA-Tat. Administering C646, CM-47, H3-CoA-20-Tat, or Lys-20-CoA-Tat to the allograft recipients restored rejection, whereas infusion of DMSO alone led to long-term Treg-dependent allograft survival (FIG. 4A). Rejection was defined as cessation of heartbeat.

In the second experiment, B6 RAG−/− mice (n=4/grp) were grafted with fully MHC-mismatched BALB/c cardiac allografts and adoptively transferred with WT B6 Teff cells (1×10⁶) alone, or WT B6 Teff cells (1×10⁶) plus C646, a p300 inhibitor (p300i). The p300i was delivered to the allograft recipients at 0.9 mg/kg/d for 14 d using Alzet pumps. In addition, WT B6 mice received normal B6 hearts (n=4/group), and were then treated with DMSO or C646. Allograft data show that p300i does not suppress Teff cell responses in vivo, and isograft data show that p300i use does not directly affect graft function (FIG. 4B).

In the third experiment, B6/RAG−/− mice (n=4/grp) were engrafted with fully MHC-mismatched BALB/c cardiac grafts and adoptively transferred with WT B6 Teff cells (1×10⁶) alone or plus Tregs (0.5×10⁶). Alzet pumps were used to deliver for 14 days DMSO (control), peptidic (Lys-CoA-Tat) or non-peptidic (C646) p300i. Whereas Tregs suppressed T cell allogeneic responses in DMSO-treated mice, concomitant p300 targeting impaired Treg rejection and restored T cell-dependent graft rejection (FIG. 4C), suggesting that Tregs are far more susceptible to inhibition by p300i than conventional T cells. Use of p300−/− Tregs was also unable to suppress Teff cells and prolong allograft survival in this adoptive transfer model.

### Example 9

### Tumor Growth Suppressed by p300 Targeting

The effects of p300 inhibition on tumor cell growth were assessed in mice in two experiments.

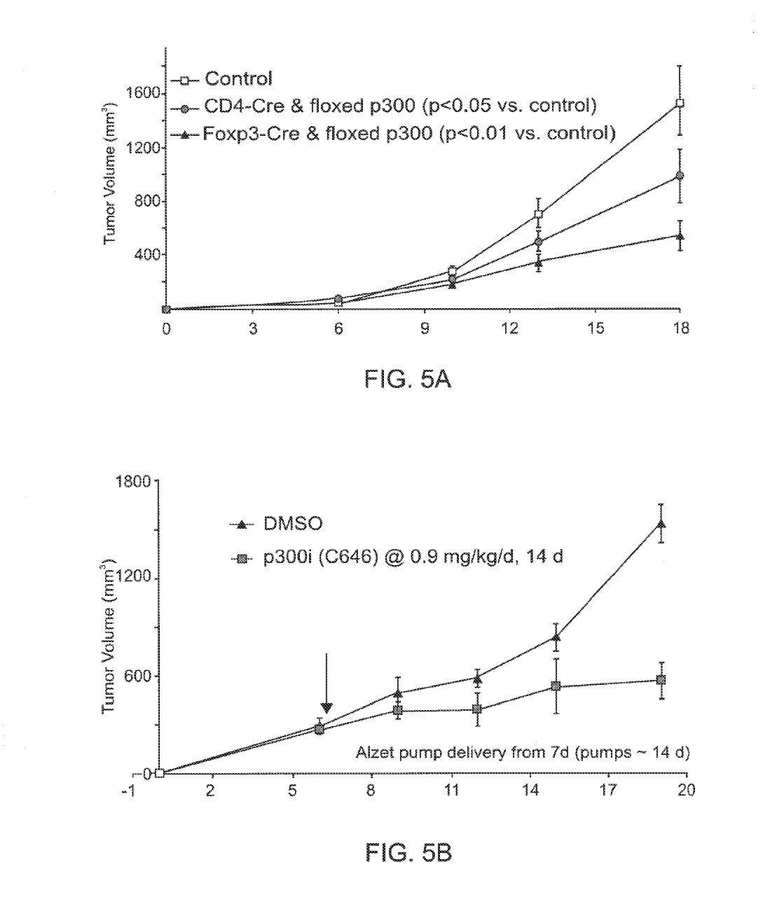

In the first experiment, p300 inhibition was achieved by p300 depletion. TC1 lung cancer cells were injected in the flanks of WT B6 mice or B6 mice in which p300 was deleted from CD4+ T cells (Treg plus Teff using CD4-Cre) or Tregs (Foxp3-Cre) (n=10/grp). Tumor volume was monitored in the mice on days 3, 6, 10, 13 and 18 post-tumor injection (FIG. 5A). p300 depletion suppressed tumor growth in the mice.

In the second experiment, p300 inhibition was achieved by using a p300 inhibitor. TC1 lung cancer cells were injected in the flanks of WT B6 mice. Starting from day 6 post-tumor injection, DMSO (control) or p300i (C646) was administered to the mice at 0.9 mg/kg/day for about 14 days via Alzet pumps. Tumor volume was monitored in the mice on days 6, 10, 12, 15 and 19 post-tumor injection (FIG. 5B). C646 suppressed tumor growth in the mice.

### Example 10

### Effects of p300i on Gene Expression and Tumor Growth

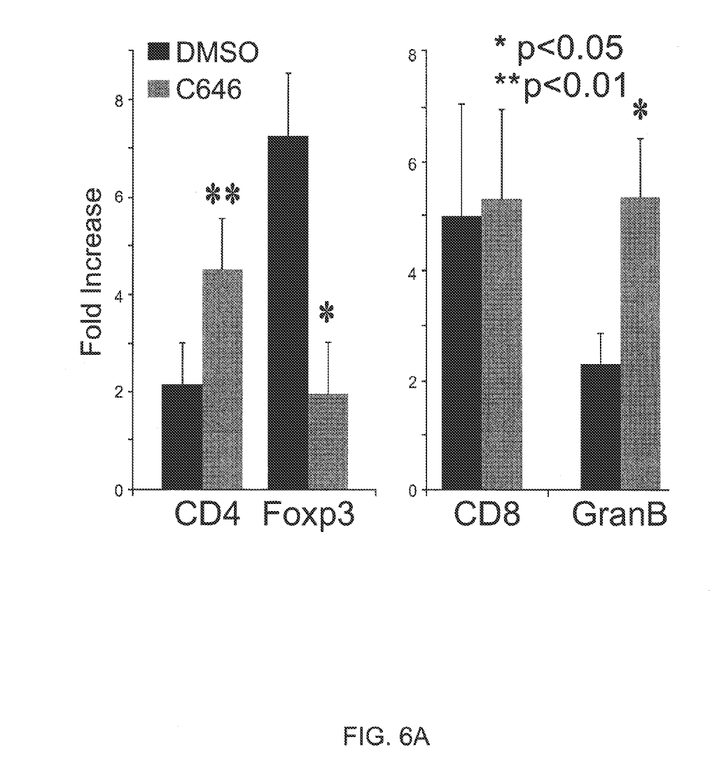

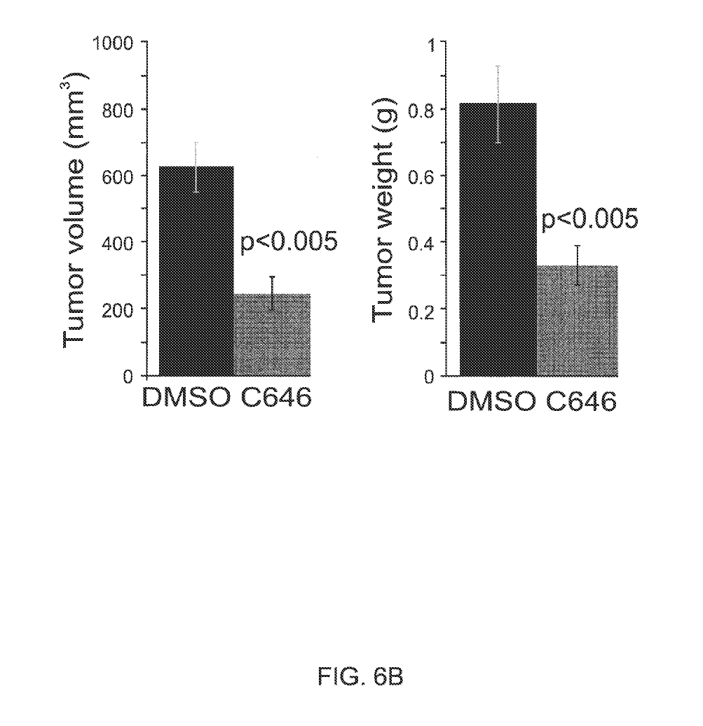

The effects of C646 (p300i) on expression of CD4, Foxp3, CD8 and Granzyme B mRNA in tumors from mice treated with DMSO or C646 were assessed. DMSO- and C646-treated mice having tumors were obtained as described in Example 9. As shown by qPCR, p300i use led to significantly increased intratumoral CD4 and granzyme B mRNA, comparable CD8 mRNA, and significantly reduced Foxp3 mRNA (FIG. 6A). These data were confirmed by immunohistology, along with increased IFN-γ production by purified CD8 (ELISPOT).

The growth of tumors in the mice was monitored by tumor volume and tumor weight (FIG. 6B). Compared with DMSO-treated mice, C646-treated mice showed significantly reduced tumor growth by weight (p<0.005) and by volume (p<0.005).

These findings indicate that p300i use results in decreased local Foxp3+ Treg infiltration and enhanced effector responses, as reflected in decreased tumor growth and upregulation of intratumoral granzyme B expression.

### Example 11

### Lack of p300i Effect on Tumor Growth in RAG−/− Mice

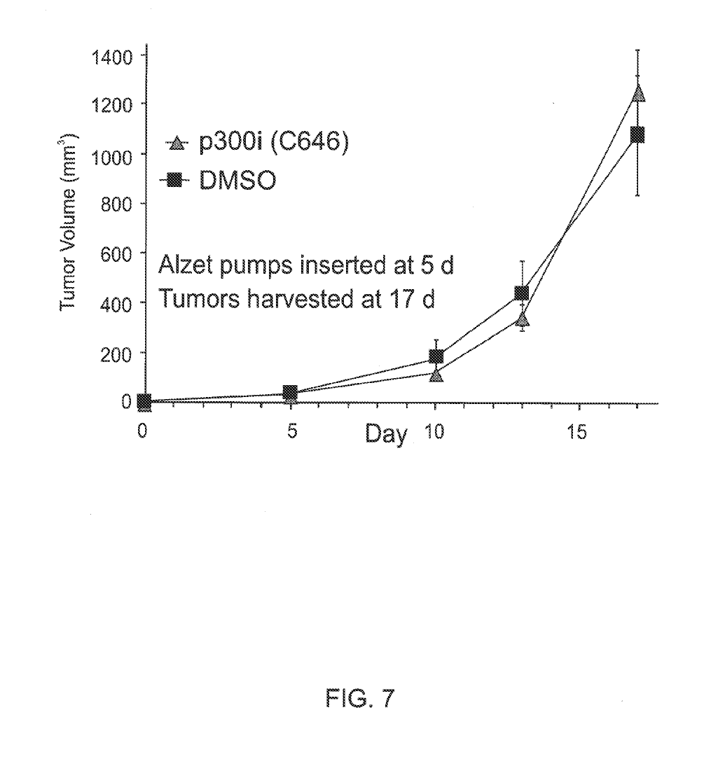

The effects of C646 on tumor growth in immunodeficient RAG−/− mice were evaluated. TC1 lung cancer cells were injected in the flanks of RAG−/− mice. Starting from day 5 post-tumor injection, DMSO (control) or p300i (C646) was administered to the mice via Alzet pumps. Tumors were harvested from the mice 17 days post-tumor injection. Tumor volume was monitored on days 0, 5, 10, 13 and 17 post-tumor injection. C646 use did not impair tumor growth in the immunodeficient RAG−/− mice (FIG. 7).

Tumors were exposed to C646 in RAG−/− mice. Decreased acetylation of histone 3 was observed in tumor extracts from C646-treated mice compared to DMSO-treated mice. No effects on acetylation of certain p300-independent targets in the tumor extracts was observed (e.g. acetylation of alpha-tubulin was unimpaired).

The data from Examples 9-11 demonstrate the ability of C646 to dampen Treg function in immunocompetent tumor-bearing hosts and enhance the ability of Teff cells to limit tumor growth.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A method for treating or preventing a Foxp3+ T regulatory cell (Treg) related disease in a subject in need thereof, comprising administering to the subject an effective amount of a pharmaceutical composition comprising an inhibitor of a histone/protein acetyltransferase (HAT).

2. The method of claim 1, wherein Foxp3+ Tregs are not depleted in the subject.

3. The method of claim 1, wherein the HAT inhibitor inhibits a function of Foxp3+ Tregs.

4. The method of claim 3, wherein the Foxp3+ Tregs are obtained from the subject.

5. The method of claim 1, wherein the HAT inhibitor does not inhibit a function of effector T cells.

6. The method of claim 5, wherein the function of the effector T cells is selected from the group consisting of T cell activation, T cell proliferation, and cytokine production.

7. The method of claim 5, wherein the effector T cells are obtained from the subject.

8. The method of claim 1, wherein the HAT is obtained from the subject.

9. The method of claim 1, wherein the HAT is selected from the group consisting of GCN5, p300/CBP-associated factor (PCAF), Myst1, Myst2, Myst3, Myst4, TIP60, p300, and CBP.

10. The method of claim 1, wherein the HAT is p300.

11. The method of claim 1, wherein the HAT inhibitor is selected from the group consisting of Lys-CoA, H3-CoA-20, C646 and functional derivatives.

12. The method of claim 1, wherein the HAT inhibitor is C646.

13. The method of claim 1, wherein the Foxp3+ Treg related disease is a solid tumor.

14. The method of claim 13, wherein the solid tumor is selected from the group consisting of lung, ovary, endometrium, cervix, breast, prostate, head, neck, esophagus, stomach, liver, pancreas, colon, and skin (melanoma) tumors.

15. The method of claim 1, further comprising administering to the subject a cancer vaccine.

16. A method for identifying an agent useful for treating or preventing a Foxp3+ T regulatory cell (Treg) related disease, comprising
(a) contacting a candidate agent with a test sample comprising Foxp3+ T regulatory cells (Tregs), and
(b) comparing a function of the Foxp3+ Tregs in the test sample with that in a control sample, wherein inhibition of the function of the Foxp3+ Tregs in the test sample when compared with the control sample indicates that the candidate agent is an agent useful for treating or preventing a Foxp3+ Treg related disease.

17. The method of claim 16, wherein the agent useful for treating or preventing the Foxp3+ T regulatory cell (Treg) related disease is an inhibitor of a histone/protein acetyltransferase (HAT).

18. A medicament useful for treating or preventing a Foxp3+ T regulatory cell (Treg) related disease in a subject, comprising an effective amount of an inhibitor of a histone/protein acetyltransferase (HAT).

19. A pharmaceutical composition for treating or preventing a Foxp3+ T regulatory cell (Treg) related disease in a subject, comprising an effective amount of an inhibitor of a histone/protein acetyltransferase (HAT).

20. A method of preparing a medicament useful for treating or preventing a Foxp3+ T regulatory cell (Treg) related disease in a subject, comprising admixing an inhibitor of a histone/protein acetyltransferase (HAT) with a pharmaceutically acceptable carrier or diluent.
